(19) United States
(12) Patent Application Publication
(10) Pub. No.: US 2026/0178989 A1
(43) Pub. Date: Jun. 25, 2026

(54) SYSTEM AND METHOD FOR AUTOMATED DEVELOPMENT OF MEDICAL DIAGNOSTIC SOFTWARE USING MACHINE LEARNING AND ARTIFICIAL INTELLIGENCE

(71) Applicant: Dmitri Krylov, Falls Church, VA (US)

(72) Inventor: Dmitri Krylov, Falls Church, VA (US)

(21) Appl. No.: 19/419,847

(22) Filed: Dec. 15, 2025

Related U.S. Application Data
(60) Provisional application No. 63/737,416, filed on Dec. 20, 2024.

(51) Int. Cl. G06N 20/20; G16H 10/60; G16H 50/20

(52) U.S. Cl. CPC G06N 20/20; G16H 10/60; G16H 50/20

(57) ABSTRACT

A method for constructing Artificial Intelligence (AI) medical diagnostic tools via computer-implemented graphical user interfaces, without the need for coding, is described. The method method utilizes automated AI algorithms (autoML) to train an AI model. It implements novel methods for data preparation, training and retraining of AL/ML models. It utilizes Federated Learning technology to make medical datasets in different hospitals available to other hospitals for constructing diagnostic tools. The invention can produce multiple medical diagnostic tools suitable for clinical use. It is intended for use by medical professionals, not requiring coding in computer languages.

### CROSS-REFERENCE TO RELATED APPLICATIONS

The present application claims the benefit of priority to U.S. Provisional Patent Application No. 63/737,416 entitled AN AI COMPUTER-IMPLEMENTED METHOD FOR NO-CODE, AUTOMATED, AUTOML DEVELOPMENT OF MEDICAL DIAGNOSTIC SOFTWARE filed Dec. 20, 2024 and U.S. Provisional Patent Application No. 63/790,000 entitled SAFE MATCH: A COMPUTER-IMPLEMENTED METHOD FOR DATA VALIDATION, HARMONIZATION, AND FILTERING IN FEDERATED LEARNING filed Apr. 16, 2025, which are incorporated herein by reference in their entireties.

### BACKGROUND OF THE EMBODIMENTS

### Field of the Embodiments

The present invention relates to computer-based medical diagnostic tool construction and use of the same.

### Description of the Existing Art

The extent of the diagnostic challenges in the U.S. is evident from the recent publication by Newman-Toker, D. E. et al. Burden of serious harms from diagnostic error in the USA. BMJ Qual. Saf. 33, 109-120 (2024), where US incidence of diagnostic errors is estimated at 6.0 M vascular events, 6.2 M infections and 1.5 M cancers. The article estimates that 795,000 people in the U.S. die or become permanently disabled each year due to misdiagnoses.

Availability of diagnostics is another critical area. A shortage of qualified medical doctors (MDs), medical offices and diagnostic tools effectively prevents many individuals from receiving a timely diagnosis and treatment. This disparity is especially prevalent in Medically Underserved Areas (MAUs). In patients with the “Diagnostic Big Three” diseases (vascular, infections, cancers) in MAUs it leads to access mortality and morbidity, in particular in cancer.

In hospitals, cancer diagnostic is typically performed using digital medical images and other data types by oncologists and radiologists. They face significant burn out, with 61% of radiologists reporting symptoms of burnout in a nation-wide study. Another, independent, study estimates the burnout rate in diagnostic radiologists at 54-72%. See Chew, F. S., Mulcahy, M. J., Porrino, J. A., Mulcahy, H. & Relyea-Chew, A., Prevalence of burnout among musculoskeletal radiologists, Skeletal Radiol. 46, 497-506 (2017) and Bailey, C. R., Bailey, A. M., McKenney, A. S. & Weiss, C. R., Understanding and Appreciating Burnout in Radiologists, Radiogr. Rev. Publ. Radiol. Soc. N. Am. Inc 42, E137-E139 (2022).

The trend is projected to increase in the future. Radiologists'burnout is caused, among other reasons, by seeing too many scans every day. This results in medical errors, exposing the healthcare team and hospitals to malpractice lawsuits with substantial costs, low patient satisfaction, and poor care delivery.

Artificial Intelligence (AI) assisted cancer detection has been shown to improve detection scores significantly. Further, AI can produce a large economic effect when used in hospitals. See Bi, W. L. et al., Artificial intelligence in cancer imaging: Clinical challenges and applications, CA. Cancer J. Clin. 69, 127-157 (2019) and Khanna, N. N. et al., Economics of Artificial Intelligence in Healthcare: Diagnosis vs. Treatment. Healthc., Basel Switz. 10, (2022).

Diagnostics is currently experiencing an explosive growth of AI tools. A large percentage of AI diagnostic tools are built to diagnose cancer. These AI tools use diverse data types including, but not limited to, computed tomography (“CT”), Magnetic Resonance Imaging (“MRI”) and positron emission tomography (“PET”). The following references provide a snapshot of the state of the art and are incorporated herein by reference in their entireties: Bi, W. L. et al. Artificial intelligence in cancer imaging: Clinical challenges and applications. CA. Cancer J. Clin. 69, 127-157 (2019); Majumder, A. & Sen, D. Artificial intelligence in cancer diagnostics and therapy: current perspectives. Indian J. Cancer 58, 481-492 (2021) and Musa, I. H. et al. Artificial Intelligence and Machine Learning in Cancer Research: A Systematic and Thematic Analysis of the Top 100 Cited Articles Indexed in Scopus Database. Cancer Control J. Moffitt Cancer Cent. 29, 10732748221095946 (2022). This extensive literature indicates that all major cancer types can be successfully diagnosed with AI tools. However, each of the cited work typically focuses on a single cancer type only.

Other diseases diagnosed with AI include stroke, pneumonia and diabetes. Currently available AI diagnostic tools typically target a single disease or a limited set of disease areas. For example, Open Source LST-AI targets lesion segmentation, an AI targets radiological diagnostic of COVID, a prostate cancer diagnostic tool that improves Gleason grading, lesions in chest radiographs are addressed by a recent AI project, and lung cancer cells were successfully identified by tissue histopathology AI. While often achieving excellent accuracy, existing AI tools are often narrow in the scope of diseases they diagnose. What is lacking is a system and method which facilitates the automated building of diagnostic tools by individuals in the medical field without the need for coding or data science skills.

### SUMMARY OF THE EMBODIMENTS

In a first exemplary embodiment, a computer-implemented method for constructing, fine-tuning and improving medical diagnostic tools responsive to user selections via one or more Graphical User Interfaces (GUIs) includes: providing access to medical record datasets for multiple individuals in digital form via one or more GUIs, wherein the medical record datasets include one or more digital images of one or more regions of interest of an individual's body; recording indications of one or more disease areas identified during review of the medical record datasets by multiple medical reviewers via the one or more GUIs in one or more regions of interest, wherein the recorded indications include at least an approximate location of the disease area within the one or more regions of interest and an identification of disease type; associating each recorded indication with a specific reviewed medical record dataset to form a prepared dataset; receiving a selection by a user via the one or more GUIs to train an ensemble of algorithms using multiple prepared datasets to automatically identify one or more disease areas and an indication of disease type in one or more regions of interest in an un-identified digital image of one or more regions of interest of an individual's body presented thereto; construct a medical diagnostic tool which includes the ensemble of trained algorithms for automatically diagnosing different disease areas and multiple disease types from new medical record datasets presented thereto; and automatically fine-tune the ensemble of trained algorithms using a plurality of automated machine learning (autoML) algorithms.

In a first exemplary embodiment, computer-implemented method for constructing, fine-tuning and improving medical diagnostic tools responsive to user selections via one or more Graphical User Interfaces (GUIs) includes: generating training data sets responsive to user inputs via the one or more GUIs, wherein the user inputs include recorded indications of one or more disease areas including approximate location of the disease area within the one or more regions of interest and disease type identified during user review of one or more digital images in medical record datasets; automatically associating each recorded indication with a specific reviewed medical record dataset to form the training data sets; receiving a selection by a user via the one or more GUIs to train an ensemble of algorithms using multiple training data sets to automatically identify one or more disease areas and an indication of disease type in one or more regions of interest in an un-identified digital image of one or more regions of interest of an individual's body presented thereto; construct a medical diagnostic tool which includes the ensemble of trained algorithms for automatically diagnosing different disease areas and multiple disease types from new medical record datasets presented thereto; receiving via the one or more GUIs additional training data sets including recorded indications of one or more disease areas including approximate location of the disease area within the one or more regions of interest and disease type identified during user review of one or more digital images in medical record datasets on a continuing basis; and receiving a selection by a user via the one or more GUIs to re-train the ensemble of trained algorithms using the additional training data sets.

### BRIEF DESCRIPTION OF THE DRAWINGS

Example embodiments will become more fully understood from the detailed description given herein below and the accompanying drawings, wherein like elements are represented by like reference characters, which are given by way of illustration only and thus do not limit the exemplary embodiments herein.

FIG. 1 depicts a high level block diagram of an automated AI constructor computer-implemented algorithm via a Graphical User Interface in accordance with an embodiment herein.

FIG. 2 depicts a block diagram of Federated Learning technology as applied to making patient medical datasets available to the invention's computer-implemented algorithms for use in building diagnostic AI tools.

FIG. 3 depicts a block diagram of data preparation of one or more datasets in accordance with an embodiment herein.

FIG. 4 depicts a process of human-assisted data validation during data preparation in accordance with an embodiment herein.

FIG. 5 depicts a system and process flow for shadowing a medical professional by a computer algorithm to capture data relevant to medical image analysis and diagnosis in accordance with an embodiment herein.

FIGS. 6A and 6B depict exemplary labeled Anatomy Maps produced by an AI/ML algorithm model diagnostic tool in accordance with an embodiment herein.

FIG. 7 depicts the construction of a Combined Density map from an Anatomy map and a Diagnostic map in accordance with an embodiment herein.

FIG. 8 depicts a workflow of Continuous Learning Technology in accordance with an embodiment herein.

FIG. 9 provides Continuous Learning Technology example in accordance with an embodiment herein.

FIG. 10 depicts an assignment of different algorithms to specific regions of the Anatomy map in accordance with an embodiment herein.

FIG. 11 depicts a schematic of hash creation for datafiles on nodes in a Federated Learning system in accordance with an embodiment herein.

FIG. 12 depicts a schematic of hash value comparison across different nodes participating in Federated Learning in accordance with an embodiment herein.

### DETAILED DESCRIPTION

In a preferred embodiment, an AI computer-implemented system and a collection of methods that provide the ability for users, e.g., clinicians and researchers, to build their own diagnostic tool(s) without the need for coding or data skills. These AI computer-implemented methods put direct control of diagnostic AI creation in the hands of medical professionals and researchers. As described further herein, the embodiments implement novel processes for data preparation, training and retraining of AL/ML models.

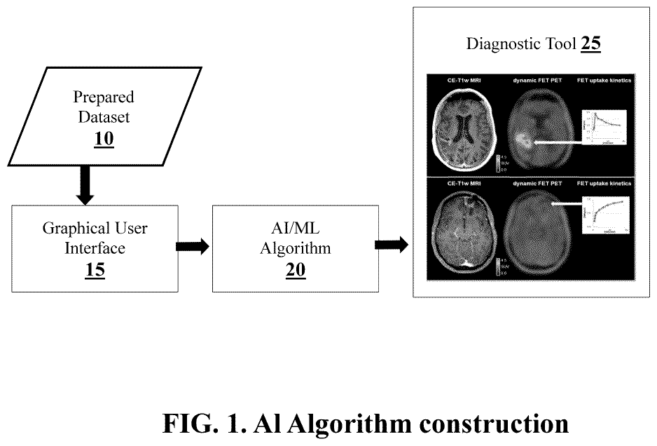

FIG. 1 depicts a high level block diagram of the workflow of the preferred embodiment, wherein prepared datasets 10 are fed into an automated AI constructor computer-implemented algorithm 20 via a Graphical User Interface (GUI) 15. The automated AI constructor computer-implemented algorithm 20 constructs an AI medical diagnostic tool 25 in an autonomous fashion, without human intervention.

As is well understood by those skilled in the art, the quality of an AI predictive algorithm/model depends on the quality and volume of data available for training and fine-tuning the algorithm/model. Large, high-quality datasets can be assembled by selecting and combining datasets from multiple medical and/or research institutions. However, a major obstacle to building such combined datasets is the inability of collecting institutions to make data available beyond their institution's boundaries. This stems, in part, from legal restrictions on sharing and disclosing PII (Personally Identifiable Information). Accordingly, a data preparation subprocess is implemented to produce the prepared datasets 10.

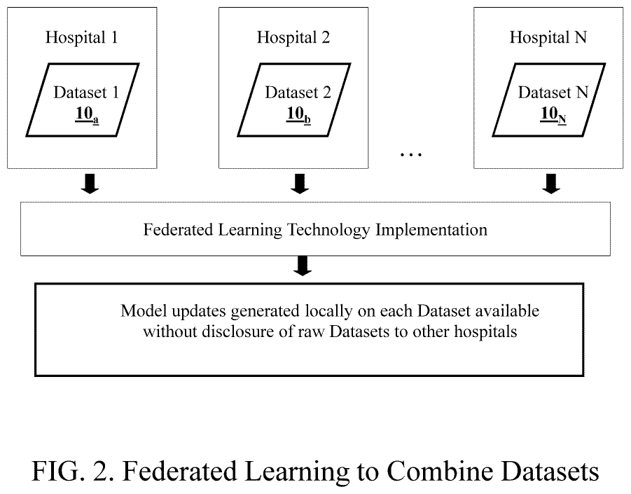

Referring to FIG. 2, Federated Learning is implemented to facilitate access to patient medical datasets 10ₐ, 10_{b} . . . 10_{N} from multiple institutions (e.g., hospitals, imaging practices, etc.) for use in building diagnostic AI tools. Using Federated Learning processes, local models are trained on each of the participating datasets. The model weights are then updated and shared across participating datasets, without sharing the raw data from medical datasets 10_{a*}, 10_{b*} . . . 10_{N*} or removing them from the original institution. Federated Learning enables training and fine-tuning of AI predictive algorithms/models on multiple datasets without disclosing them beyond institutional boundaries. Datasets available via a Federated Learning computer implementation are used to train and fine-tune AI predictive algorithms/models residing locally at the institutions in the process of constructing computer-implemented diagnostic tools.

Alternatively, one skilled in the art will appreciate that for implementations wherein datasets 10ₐ, 10_{b} . . . 10_{N} can be shared across nodes (e.g., institutions) and Federated Learning is not required, training of the AI/ML model described below may occur directly, using the prepared datasets, including the underlying data. Similarly, data labeling and reviews by experts may also occur across datasets and is not limited.

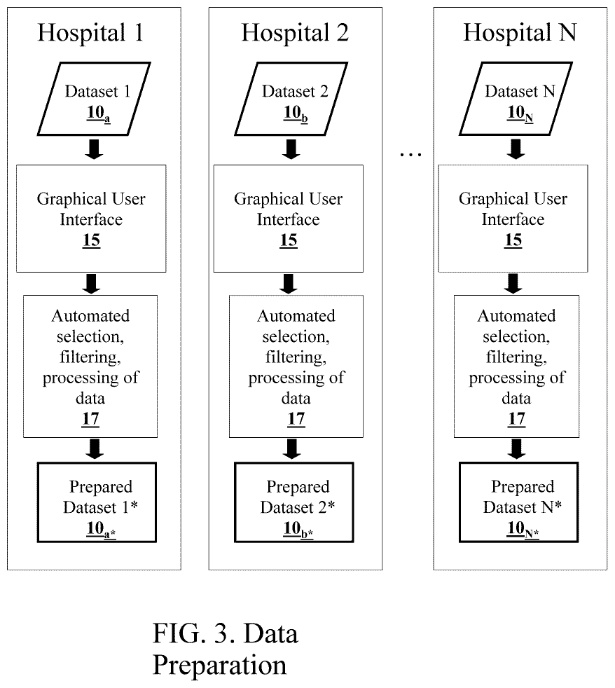

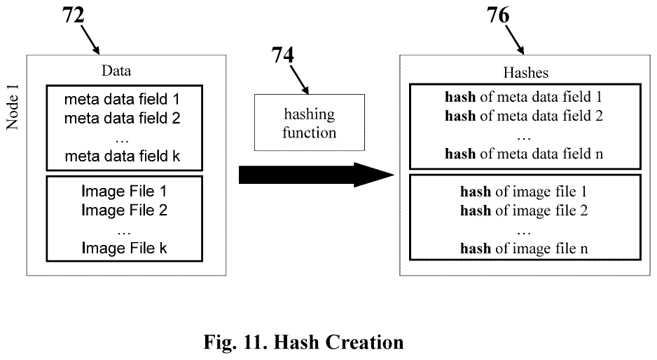

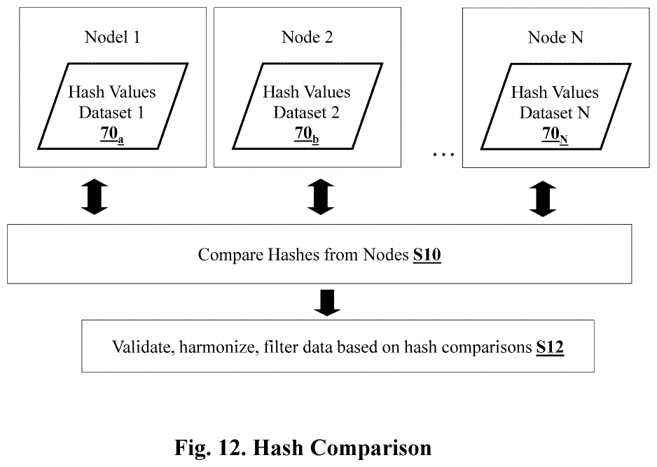

Quality of data used for model training is a key to model accuracy. Accordingly, referring to FIG. 3, the datasets 10ₐ, 10_{b} . . . 10_{N} are further filtered by an automated computer-implemented algorithm 17 to produce prepared datasets 10_{a*}, 10_{b*} . . . 10_{N*}. In a preferred embodiment, duplicated, incomplete and/or erroneous data entries are filtered out by the algorithm which is described further herein below with reference to FIGS. 11 and 12 and referred to generally herein as “Safe Match”.

Referring to FIGS. 11 and 12, in FIG. 11 meta data fields, data descriptors, image files or other relevant fields are obtained from the data 70 on each of the federated nodes (e.g., hospitals) and a computer cryptographic hash function 72 is applied thereto. In a given computer implementation, the hash function can be of several different types including, but not limited to SHA, BLAKE, MD, BLAKE or another cryptographic function producing a cryptographic hash of the digital data. The result of applying the hash function is a set of hash values corresponding to the meta data fields, data descriptors, image files or other relevant fields 74. Alternatively, the hash function could be a non-cryptographic hash including, but not limited to: a fuzzy hash function, cyclic redundancy check, checksum, universal hash function, a keyed or unkeyed hash function producing a non-cryptographic hash of the digital data. Alternatively, or in addition thereto, pixels of the digital image itself are taken as a sequence of bits and hashed and the hashed bit sequence is compared to see if the digital image is blank and to filter out some other problems. All of this can be done without disclosing PII, merely by looking at the hashes.

FIG. 12 depicts a block diagram of the comparison of the hash values for equality, partial or complete. This comparison S10 can be performed across the hashes of medical digital images and/or across the hashes of meta data fields, data descriptors or other relevant fields. In the case of fuzzy hashing, if values are found to be exactly equal, the corresponding medical digital images, meta data fields, data descriptors or other relevant fields are also considered equal. If the hash values are found to be partially equal, the corresponding meta data fields, data descriptors or other relevant fields are also considered to be partially equal or close in value.

Equality of hashes for images means duplication, and duplicate images are discarded from the training set. Equality of meta fields may mean duplication, if the values of some meta data are equal, and in this case they are also discarded. Equality in the names of the meta fields means that the data and/or in other types of values of meta data fields indicates that the datasets are comparable and these are selected for model training.

The results of the comparison enable to validate, harmonize and filter data based on the results of the comparison S12. Due to the one-way nature of the hash function transformation, no data is exposed outside of the originating node. Nodes with data with partially or completely equal meta data fields can be selected for further use in training the AI/ML models as described herein.

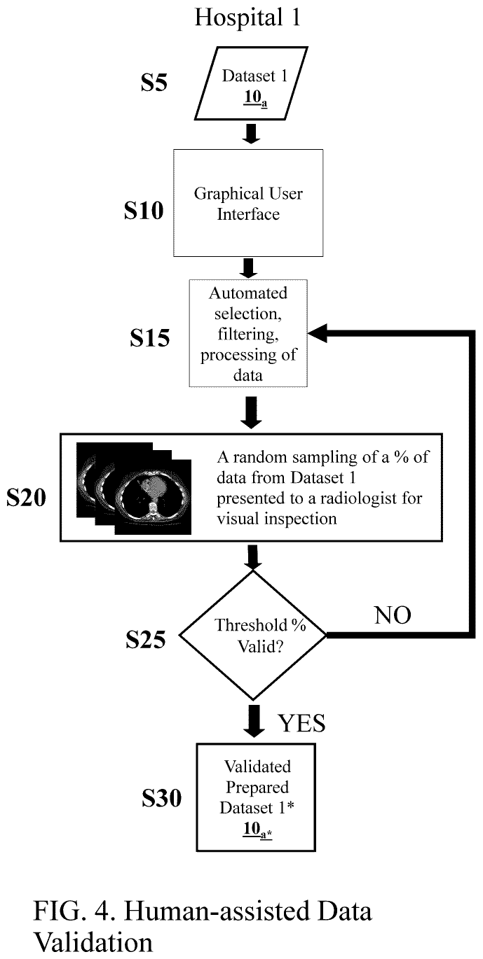

Further, an additional data filtering step with human input is implemented in the preferred embodiment as shown in FIG. 4. At S5 each dataset 10 is input to the system via GUI S10 and is subject to an initial filtering and selection process via automated algorithms S15, including Safe Match. Next, a random sampling of the data is presented to a domain expert S20. In a specific example, the data could include medical images and the domain expert is a radiologist that reviews the sampling for verification S25. In order to reduce the radiologist's workload, only a representative number of the medical digital images are shown for human inspection, sufficient to produce a confidence interval of, for example, 95-98%. If the data passed human inspection within this confidence level, it is labeled as a validated prepared dataset 10* and may be used for model training S30. Otherwise, the entire dataset 10 is re-filtered and re-validated automatically again at S15 with the strictness of validation parameters of the selection algorithm increased. Steps S20 and S25 are repeated until a desired number of medical digital images passes validation, or it is found that the dataset does not have enough valid medical digital images.

Large dataset with thousands of images or more can be validated quicker this way, as the sampling only requires hundreds or up to a thousand images, provided the dataset is homogeneous.

Accordingly, one or more of the validated prepared dataset 10* may be processed and encoded for training and/or fine-tuning the AI predictive algorithm/model. This is accomplished with minimal human interaction, in every case using graphical User Interface and without writing computer code.

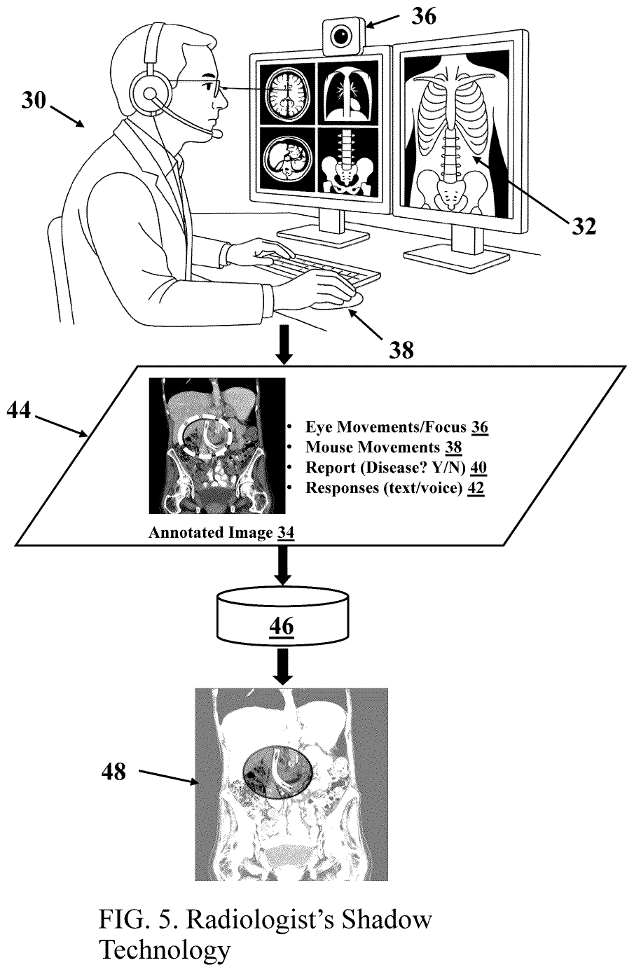

Further to the imaging domain embodiment exemplified herein, digital image data within the original datasets 10ₐ, 10_{b} . . . 10_{N} is collected in an on-going fashion as medical experts are reviewing images to obtain high quality labeled training data. Referenced herein as “Radiologist's Shadow” and referring to FIG. 5, a system and process records a radiologist's 30 (or other clinician's) behavior when they are working with medical digital images (or other medical data). When enabled, one or more unlabeled (unsegmented) digital images 32a-x is presented to the radiologist. Images may come from a patient with disease or with no disease and display signs of disease or no disease.

In one embodiment, the presented images may be previously selected for labeling as a training set of medical images. Alternatively, the process can function in the background in order to continuously build the database of images, while the radiologist is performing routine tasks of analyzing incoming patients'medical digital images.

The radiologist can turn Radiologist's Shadow on or off in order to feel comfortable in their work.

The radiologist or clinician is asked to find diseased areas, draft a report on the image and state whether the image has or does not have signs of a particular disease. If determined to illustrate disease, the radiologist is asked to mark (segment, trace) the area(s) 34 they identify as indicative of disease. The radiologists will further define the borders of the diseased area, calculate its sizing and volume and define its density. They will perform other task routinely done by radiologists analyzing a medical image.

While the radiologist is analyzing an image, their eye ball movements are tracked via a digital camera 36 and recorded, computer mouse 38 movements on the computer screen are tracked and recorded, and their reports 40 and the responses to voice or textual prompts 42 are recorded and stored for further encoding and use in training the AI/ML algorithm. The eye ball movement is mapped onto the areas of the images showing on the screen where the radiologist is focusing their gaze. This mapping is further assisted by the track of mouse and cursor movements over the radiologist's computer screen and their use of magnification of an area of image, changes of view angle, and use of other visual and analytical features while they are analyzing the image.

Several radiologists and/or other clinicians are asked to analyze the same set of training medical images. All of their eye ball movements, computer mouse movements on the computer screen, and their responses to voice or textual prompts and other use data are recorded and stored with the individual images in data file(s) 44 in a database 46.

A computer implemented process accesses the recorded and stored data and averages it over several radiologists by using mean values coordinates of where the eyeballs of the radiologists are focused and where their computer mouse dwells. These coordinates can exist in 2-dimensional (2D) or 3-dimensional (3D) space based on the dimensionality of the medical image. These averaged areas produce a 2D or 3D map of the medical image, wherein more weight is assigned to areas where the radiologist's eyeball and mouse spend more time and less weight where they spend less time. This map, with weights for all areas of the image, is called a Diagnostic Density Map 48.

Diagnostic Density Map 48 is further normalized to one scale to make it usable across different images.

The resultant weights for all areas of an image placed on the Diagnostic Density Map 48 are used in scaling the input image data for training AI/ML model. This novel technique allows for reduced AI/ML model training time and increased accuracy of the produced model.

Medical images typically display a part of the human body or an organ. Cancer cells and other disease areas localize unevenly in the human body. For example, colorectal cancer cells are typically found in the column or rectum. While metastatic cells can spread to the lymph nodes and other tissues and organs, their localization still follows a non-uniform pattern. For instance, in colorectal cancer metastases are found in the peritoneal space in an estimated 10-30% of the time.

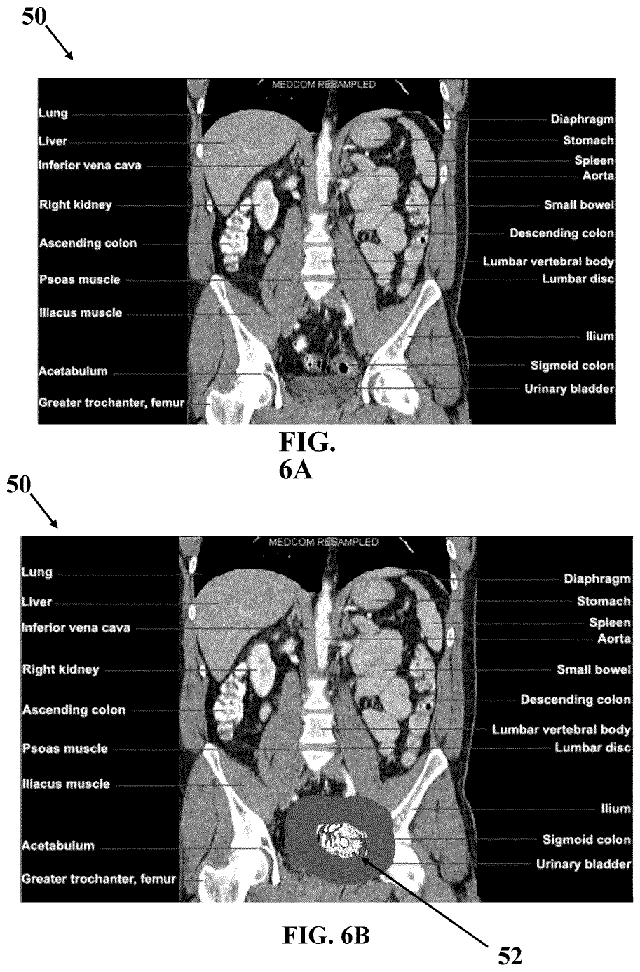

Referring to FIGS. 6A and 6B, the present embodiments use an Anatomy Map 50 to simplify the identification and characterization of diseased areas, such as cancer cells in the human body.

A pre-existing computer algorithm capable of identifying human organs and tissues in medical digital images is used to produce the Anatomy Map for a given image. The output of the pre-existing computer algorithm is the spatial coordinates of organs and tissues for a given medical digital image. It is called the Anatomy Map of an image.

The Anatomy Map also carries a weight for each pixel or voxel of the medical digital image. Lower weights are assigned to areas where a particular disease is unlikely to be found, according to existing cancer research, while higher weights indicate a more likely localization of the diseased area.

FIGS. 6A and 6B are exemplary anatomy maps generated by the pre-existing computer algorithm. While FIG. 6A is a base CT scan Anatomy Map with primary organs and tissues identified and no highlighted areas of disease, FIG. 6B indicates a case of colorectal cancer, wherein the colon and the rectum are assigned higher weights, when identifying the primary tumor 52 on the Anatomy Map 50. The peritoneal space and the lymph nodes are assigned a higher weight (score) for identifying metastases of such tumor, while all the other parts of the scan are assigned a lower score.

When presenting the AI/ML model images for organ/tissue identification and generation of an Anatomy Map 50, the accuracy of body organ/tissue identification depends on the quality of the medical digital image (e.g., 32) and the proficiency of the computer algorithm used. The reliability of organ/tissue identification by the computer algorithm on a presented image(s) will be reflected by a numerical score.

If organs/tissues cannot be identified correctly because of poor image quality, deficiencies in computer algorithm, or any other reason, and the resultant reliability score is below a cut-off value, this identification is not to be used. Alternatively, a coarse division of the image into areas may be applied. Such a division will be produced based on the sizes of body areas in a typical human anatomy. For example, an abdominal digital image is divided into upper left, upper right, lower left, and lower right quadrants.

The scoring matrix, either from organ/tissue identification or from coarse anatomy mapping, with scores of where a particular disease localizes, is used both during the training of the AI/ML model and during the diagnosis.

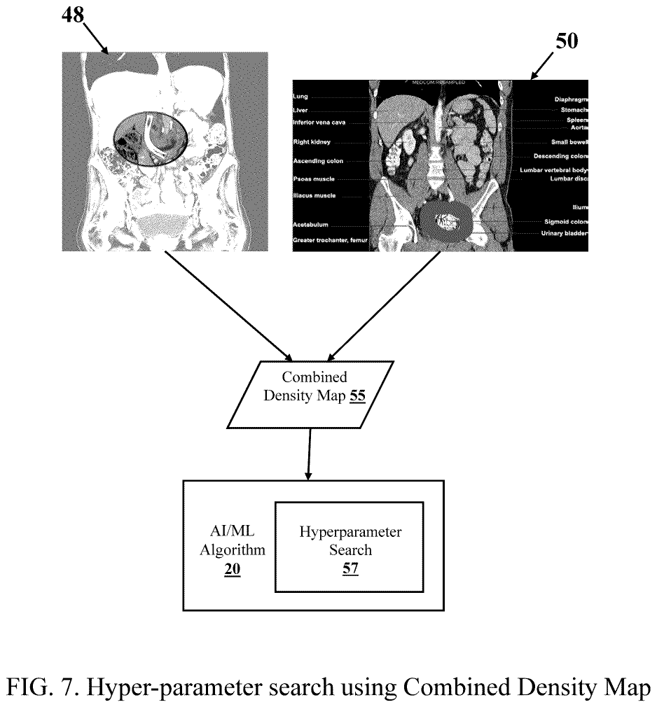

As is well known to those skilled int he art, successful training of an AI/ML model depends on an optimal combination of the model hyper-parameters. Typically, there are several hyper-parameters and their numerical values vary over a considerable range, making an optimal combination search time consuming and non-trivial. In a preferred embodiment, a technique of searching through the space of hyperparameters called Combined Density Map Gradient Decent is used herein and a high level workflow is partially illustrated on FIG. 7 wherein the AI/ML model performs a hyper-parameter search using a Combined Density Map. A Combined Density Map 55 is the result of an overlay of Diagnostic Density Map 48 and an Anatomy Map 50 pertaining to the same original medical digital image. The Combined Density Map 50 is used in defining the loss function during the hyper-parameter search 57.

This search follows the traditional gradient descent approach but the loss function is not calculated solely based on the segmentation of the medical digital image, which entails using pixel or voxel coordinates of labeled regions. Rather, it is calculated based on the normalized Diagnostic Density Map weight values combined with the normalized Anatomy Map weight values and further combined with segmentation, when available. Higher scores are assigned to hyper-parameter combinations that produce a model selecting high density regions on the Diagnostic Density Map and the Anatomy Map.

Using Diagnostic Density and Anatomy Maps as well as their combination, a Combined Density Map, is particularly useful for Un-supervised Machine Learning. As is well known for those skilled in the art, currently the majority of medical image data remains unlabelled (unsegmented) by radiologists and the ability to segment the images in an automated way can be valuable for training AI/ML models using an automated labeling (segmentation) method.

In case of Supervised Learning, this approach of using a Diagnostic Density Map and an Anatomy Map in another embodiment can be combined with radiologist's labeling, tracings, segmentations and reports for the same medical digital images, if radiologist's labeling is available

Moreover, this approach to model training saves time and it is more likely to find global minimum when a complex set of hyper-parameters is used. Additionally, hyper parameter optimization may be performed by random search, grid search, Hyperband, and Bayesian optimization using probability density trees, and random forests; ensembles of neural networks constructed by evolutionary algorithms and network morphism; BOHB for constructing per-processors, training optimizer, and constructing an optimal network architectures.

Once the algorithm/model reaches a desired accuracy, as measured by the rates of positive and false negatives, Area Under the Curve and other metrics when run on the validation dataset, it will build into a GUI for the daily use of medical professionals, clinicians, radiologists and researchers to diagnose a disease.

This GUI will enable uploading of digital scans, laboratory tests and the plurality of other digital data, including but not limited to angiography, electrocardiogram (ECG), magnetic resonance imaging(MRI), computerized tomography (CT), positron emission tomography (PET), echo cardiogram, ultrasound scan, X-ray scan can be analyzed, all in a GUI, without coding in computer programming languages.

After a trained model is deployed in clinical settings and is used to assist in patient diagnostics and treatment, the post-diagnosis and post-treatment patient data is recorded and stored (referred to herein collectively as “post-diagnosis data”). This post-diagnosis data includes follow up digital medical images, lab test results, and, importantly, outcomes for patient after they are diagnosed and treated.

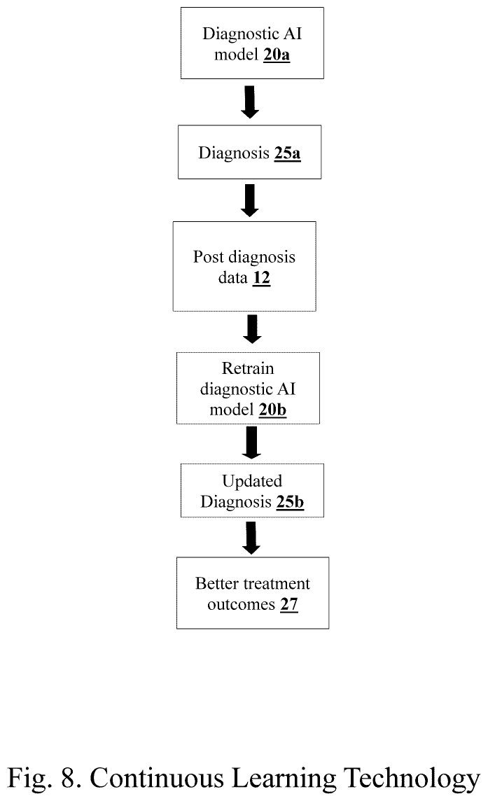

Referring to FIG. 8, post-diagnosis data is used to perform continued re-training (“continuous learning”) of the AI/ML model to improve outcomes. In FIG. 8, a first AI/ML model instantiation 20a trained on pre-diagnosis data determines a first diagnosis 25a. In this example, based on this first diagnosis 25a, treatment is prescribed and post diagnosis data 12 is used to retrain the first AI/ML model 20a resulting in second AI/ML model instantiation 20b. The result of this retraining might change the diagnosis 25b and potentially improve patient outcomes 27.

As is understood to those skilled in the art, post-diagnosis data may be split into a training and validation sets and encoded for use in re-training an AI/ML model. The AI/ML model is retrained with an updated dataset consisting of original data from the old training set combined with the post-diagnosis encoded data. The retrained AI/ML model is tested on the old and the new validation sets and the performance of the retrained AI/ML model is evaluated. If it is found that its performance has improved, in particular, that it diagnoses disease states that were previously missed, without losing its performance on the old dataset, it is assumed that the AI/ML model now performs better and the retrained AI/ML model becomes the production model.

A diseased area, such as a mass of cancer cells, can be missed on the first digital scan of the patient because it is too small and/or its density is not visibly different from the surrounding tissue for a human. On subsequent scans, post-diagnosis and post-treatment, this area may increase in the patient in size and density. Accordingly, mapping this increased area to the initial scan and retraining the AI/ML model until it identifies this area as diseased in the initial scan will greatly increase the sensitivity of the diagnostics.

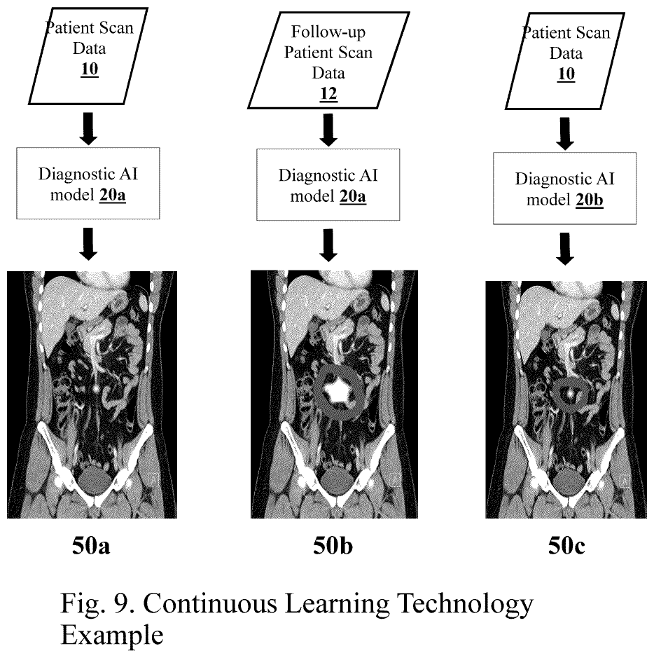

Take the example of FIG. 9 of three Anatomy Maps 50a, 50b and 50c. The first anatomy map was generated by a first AI/ML model 20a based on first patient scan data 10 and identified no disease in Anatomy Map 50a. Follow-up (post-diagnosis) patient scan data 12 for the same patient was presented to first AI/ML model 20a and a tumor is identified in resulting Anatomy Map 50b. After the first AI/ML model 20a is retrained using both first patient scan data 10 and post-diagnosis patient scan data 12, the second AI/ML model 20b is able to identify the tumor from the original first patient scan data 10 in Anatomy Map 50c.

Each time an AI/ML model is retrained, its other performance characteristics are monitored and confirmed. In particular, the retraining of the model should not result in over-diagnosis and higher false-positives. An optimal balance between sensitivity to previously missed diseased areas and acceptable false-positive rate is ensured during retraining.

Now the retrained AI model is used and it produces better diagnostic assistance. Continuous learning operates at all times patients are diagnosed. Patient outcomes and new patient studies are recorded and saved for another round of Continuous Learning model retraining.

In the medical diagnostic domain, the anatomy of medical digital images is used to determine the weights for the autoML ensemble of models used in the AI/ML model. As discussed herein, the training medical digital images are divided into several regions according to the organ or tissue they represent. The actual number of regions depends on the body part of the medical digital image. Each region is tested with each and all autoML algorithms and the best individual algorithm performer is determined per region. AutoML ensemble is then combined in an organ-or tissue-specific manner.

An algorithm that predicts disease best for a given organ or tissue is designated as that organ/tissue's Authority Algorithm. In an autoML ensemble, each organ/tissue has an Authority algorithm and it is assigned to the part of the image (the organ or tissue), where it is bound to the organ(s) or tissue(s) where it performs best.

It is established in the literature that different kinds of AI/ML features work better for a given disease and image type. Designating Authority Algorithms allows the AI/ML model to have multiple kinds of feature selections on a single image depending on the different organ/tissue identified in the image, thus increasing the performance of the AI/ML algorithm.

Combinations of autoML algorithms with weights can be applied to a region of the medical digital image, if it is found that a single algorithm does not result in a desired performance. Two or more autoML algorithms can then be combined with weights when diagnosing a particular region.

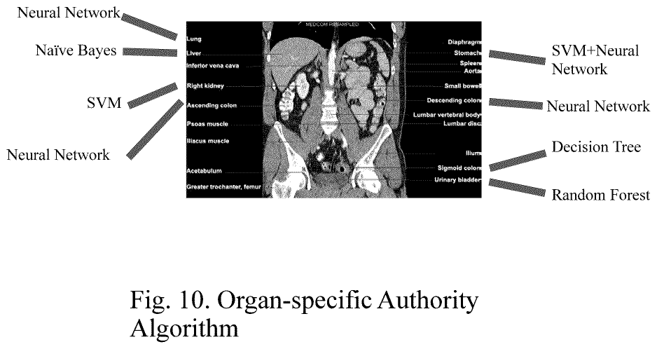

Finally, the processed and encoded datasets are applied to train an AI predictive algorithm/model to make a diagnoses. The datasets are divided into a training set and a validation set. The process of training and fine-tuning is performed by an automated workflow; wherein multiple iterations of training or fine-tuning are conducted until a desired accuracy of the resulting predictive algorithm/model is reached as measured by its performance on a validation dataset. The algorithms for the predictive model will contain one or a combination of Artificial Neural Networks, Naïve Bayes Classifier Algorithm, K Means Clustering Algorithm, Support Vector Machine Algorithm, Linear Regression, Logistic Regression, Decision Trees, Random Forests, Nearest Neighbors as shown in FIG. 10. The construction of the predictive model is accomplished by the invented workflow. This workflow enables an autonomous creation of a medical diagnostic tool. It is accomplished in a graphical User Interface, without coding in computer programming languages.

Once the AI/ML model is validated, it is integrated into a computer-implemented algorithm that enables clinicians to analyze incoming patients'medical digital images and other medical data. Once uploaded from a medical scanner or otherwise entered into the computer-implemented algorithm, the data is analyzed by the AI predictive diagnostic tool and a likely diagnosis is issued. It can include an area or areas on a computer digital image pointed out graphically as a disease area. It can also include textual prompts listing out the discovered disease area or areas and the likely type of pathological changes in the tissue, associated with them. It can also include an indication of a fragment or fragments of another data type identified as an indication of a disease, with or without textual prompts to list out this fragment or fragments with the corresponding disease.

The AI predictive diagnostic tool, constructed by our invented computer-implemented algorithm, can be further fine-tuned and improved using the same invented computer-implemented algorithm. In order to do that, a fine-tuning or improvement workflow is launched in a graphical User Interface.

The fine-tuning and/or improvement of the tool is conducted by retraining the algorithm/model using the same or a different dataset, with or without hyper-parameter optimization, all in our invented automated computer-implemented algorithm, without human coding and solely in graphical User Interface.

The result of the fine-tuning and/or improvement of the tool is its higher performance as measured by the rates of positive and false negatives, Area Under the Curve and other metrics when run on the validation dataset.

The resultant fine-tuned and/or improved tool can be used in the invented graphical User Interface for the daily use of medical professionals, clinicians, radiologists and researchers to diagnose a disease.

The embodiments described herein provide a versatile AI computer-implemented method for building AI diagnostics tools. While existing commercial AI diagnostics have been developed by software engineers and data scientists, the present embodiments put development in the hands of the users and empower health care professionals and scientists to construct and improve diagnostic tools directly. This is important, because MDs and scientists better understand the requirements of automated diagnostics. They also understand the data and the diagnostic strategies better than anyone else. With the method described herein, the diagnostic tool development cycle is greatly shorten, while the quality of the tools improves due to better domain knowledge and the ability to improve the tools at clinical settings. Further, the method described herein can shorten the development cycle and reduce costs for commercial development of AI diagnostic tools.

1. A computer-implemented method for constructing, fine-tuning and improving medical diagnostic tools responsive to user selections via one or more Graphical User Interfaces (GUIs) comprising:
providing access to medical record datasets for multiple individuals in digital form via one or more GUIs, wherein the medical record datasets include one or more digital images of one or more regions of interest of an individual's body;
recording indications of one or more disease areas identified during review of the medical record datasets by multiple medical reviewers via the one or more GUIs in one or more regions of interest, wherein the recorded indications include at least an approximate location of the disease area within the one or more regions of interest and an identification of disease type;
associating each recorded indication with a specific reviewed medical record dataset to form a prepared dataset;
receiving a selection by a user via the one or more GUIs to
train an ensemble of algorithms using multiple prepared datasets to automatically identify one or more disease areas and an indication of disease type in one or more regions of interest in an un-identified digital image of one or more regions of interest of an individual's body presented thereto;
construct a medical diagnostic tool which includes the ensemble of trained algorithms for automatically diagnosing different disease areas and multiple disease types from new medical record datasets presented thereto; and
automatically fine-tune the ensemble of trained algorithms using a plurality of automated machine learning (autoML) algorithms.

2. The computer-implemented method of claim 1, wherein the recorded indications further include segmentation features of the digital images identified by one or more of the multiple medical reviewers.

3. The computer-implemented method of claim 1, wherein recording indications of one or more disease areas identified includes one or more of tracking of computer mouse movement in the form of cursor location by the one or more medical reviewers during review of the digital images and tracking of eye ball movements in the form of gaze of the one or more medical reviewers during review of the digital images, wherein the tracking includes recording approximate coordinates of cursor location and gaze on a displayed digital image and approximate time spent at the approximate coordinates on the displayed digital image.

4. The computer-implemented method of claim 1, wherein recording indications of one or more disease areas identified includes recording of one or more of voice or typed text prompts from one or more medical reviewers during review of the digital images.

5. The computer-implemented method of claim 1, wherein the plurality of autoML algorithms are selected from the group including, but not limited to: hyper-parameter optimization by random search, grid search, Hyperband, and Bayesian optimization using probability density trees, and random forests; ensembles of neural networks constructed by evolutionary algorithms and network morphism; Bayesian Optimization and Hyperband (BOHB) for constructing pre-processors, training optimizer, and constructing optimal network architectures.

6. The computer-implemented method of claim 1, wherein the multiple algorithms are selected from the group including, but not limited to: Artificial Neural Networks, Naïve Bayes Classifier Algorithm, K Means Clustering Algorithm, Support Vector Machine Algorithm, Linear Regression algorithm, Logistic Regression algorithm, Decision Trees, Random Forests, and Nearest Neighbors.

7. The computer-implemented method of claim 1, wherein the medical record datasets comprise one or more of medical Electronic Health Record (EHR) types, laboratory test results and other digital data selected from but not limited to the group consisting of angiography data, electrocardiogram (ECG) data, magnetic resonance imaging(MRI) data, computerized tomography (CT) data, positron emission tomography (PET) data, echocardiogram data, ultrasound data, and X-ray data.

8. The computer-implemented method of claim 1, wherein a combined group of steps including providing access to medical record datasets, recording indications, forming prepared datasets and training multiple algorithms using the recorded indications occur in multiple, separate instances to secure access to the medical record datasets within independent institutional networks; and
further wherein individual training results from each of the separate instances of training of the multiple algorithms on the prepared datasets are used in constructing the diagnostic tool.

9. The computer-implemented method of claim 8, wherein instances of the combined group of steps are continuously repeated for new and updated medical record datasets for multiple individuals; and
further comprising updating the constructed diagnostic tool to improve automatically diagnosing one or more diseases using the continually updated individual training results from each of the separate repeated instances of training of the multiple algorithms.

10. The computer-implemented method of claim 1, further comprising:
filtering the prepared datasets to identify incomplete, invalid and duplicate prepared datasets to prior to use in training the multiple algorithms.

11. The computer-implemented method of claim 10, further comprising:
inspecting, by a medical inspector, a random sampling of the prepared datasets to visually confirm accuracy of the recorded indications, wherein if a threshold percentage of the random sampling are confirmed as accurate, the prepared datasets are validate for use in training the multiple algorithms.

12. The computer-implemented method of claim 10, further comprising:
securing access to the medical record datasets, wherein the securing includes selecting at least one of one or more metadata fields and one or more digital images for the medical record datasets and applying a hashing function to the at least one of one or more identified metadata data fields and one or more digital images to produce hashes of the selected at least one of one or more identified metadata data fields and one or more digital images for the medical record datasets.

13. The computer-implemented method of claim 12, wherein filtering the prepared datasets includes comparing the hashes of the selected at least one of one or more identified metadata data fields and one or more digital images.

14. The computer-implemented method of claim 1, wherein the one or more regions of interest are selected from the group consisting of tissue type and organ type; and
further wherein different ones of the multiple algorithms are trained to identify one or more disease areas in different tissue and organ types.

15. The computer-implemented method of claim 3, further comprising:
generating, by an imaging algorithm, an anatomy map for each digital image in the medical record datasets, wherein the anatomy maps visually display tissue type and the organ type anatomically using one of pixel weight values and voxel weight values; and
updating the anatomy map responsive to the tracking to form a density map for each digital image, wherein the one of pixel weight values and voxel weight values are increased or decreased in accordance with tracked approximate time spent at each approximate location on each digital image.

16. The computer-implemented method of claim 15, wherein fine-tuning the ensemble of algorithms includes hyper-parameter optimization comprising making loss function calculated using a combined density map more accurate, wherein the combined density map is the density map for each digital image overlaid on the anatomy map for each digital image.

17. The computer-implemented method of claim 15, wherein training the ensemble of algorithms includes making loss function calculated using a combined density map more accurate, wherein the combined density map is the density map for each digital image overlaid on the anatomy map for each digital image.

18. A computer-implemented method for constructing, fine-tuning and improving medical diagnostic tools responsive to user selections via one or more Graphical User Interfaces (GUIs) comprising:
generating training data sets responsive to user inputs via the one or more GUIs, wherein the user inputs include recorded indications of one or more disease areas including approximate location of the disease area within the one or more regions of interest and disease type identified during user review of one or more digital images in medical record datasets;
automatically associating each recorded indication with a specific reviewed medical record dataset to form the training data sets;
receiving a selection by a user via the one or more GUIs to
train an ensemble of algorithms using multiple training data sets to automatically identify one or more disease areas and an indication of disease type in one or more regions of interest in an un-identified digital image of one or more regions of interest of an individual's body presented thereto;
construct a medical diagnostic tool which includes the ensemble of trained algorithms for automatically diagnosing different disease areas and multiple disease types from new medical record datasets presented thereto;
receiving via the one or more GUIs additional training data sets including recorded indications of one or more disease areas including approximate location of the disease area within the one or more regions of interest and disease type identified during user review of one or more digital images in medical record datasets on a continuing basis; and
receiving a selection by a user via the one or more GUIs to re-train the ensemble of trained algorithms using the additional training data sets.
